(19) United States
(12) Patent Application Publication
(10) Pub. No.: US 2024/0261267 A1
(43) Pub. Date: Aug. 8, 2024

(54) METHODS FOR TREATING MULTIPLE SCLEROSIS

(71) Applicant: Vanda Pharmaceuticals Inc., Washington, DC (US)

(72) Inventors: Allitia DIBERNARDO, Raritan, NJ (US); Maria AIT-TIHYATY, Ontario (CA); Frank WIEGAND, Raritan, NJ (US); Lindsey LAIR, Raritan, NJ (US); Zaid Serhal SAAD, Raritan, NJ (US); Ritobrato DATTA, Raritan, NJ (US); Hartmuth KOLB, Raritan, NJ (US)

(21) Appl. No.: 18/565,690

(22) PCT Filed: Nov. 4, 2022
(86) PCT No.: PCT/EP2022/080823

§ 371 Date: Nov. 30, 2023

Related U.S. Application Data
(60) Provisional application No. 63/276,147, filed on Nov. 5, 2021.
(60) Provisional application No. 63/342,825, filed on May 17, 2022.

(51) Int. Cl. A61K 31/426; A61K 31/397; A61P 25/28

(52) U.S. Cl. CPC A61K 31/426; A61K 31/397; A61P 25/28

(57) ABSTRACT

The disclosure relates to methods of preserving myelination of axons in a human subject having a demyelinating disease by administration of an effective amount of a monoselective S1P receptor modulator, such as ponesimod.

### CROSS-REFERENCE TO RELATED APPLICATIONS

This application claims the benefit of U.S. Provisional Patent Application Nos. 63/276, 147, filed Nov. 5, 2021, and 63/342,825, filed May 17, 2022, the disclosures of each of which are incorporated by reference herein.

### TECHNICAL FIELD

The present disclosure relates to methods of preserving myelination of axons in a human subject having a demyelinating disease.

### BACKGROUND

Multiple sclerosis (MS) afflicts approximately 400,000 people in the United States and 2.5 million worldwide. MS is an inflammatory disease in which myelin sheaths around the axons of the brain and spinal cord are damaged. In MS as well as other demyelinating diseases, autoimmune inflammatory attack against myelin and oligodendrocytes causes demyelination. The thinning or loss of myelin surrounding axons impairs the ability of the axons to effectively conduct signals and results in progressive neuronal damage.

Remyelination is the process by which new myelin sheaths are generated around axons. Remyelination can occur following the loss of myelin in diseases such as MS, thus restoring neurological function to axons. However, although remyelination can occur in the early stages of MS, oligodendrocytes are unable to completely rebuild the myelin sheath, and repeated inflammatory attacks ultimately lead to fewer effective remyelinations until plaques build up around the damaged axons. A primary cause of remyelination failure is the progressive inability of somatic oligodendrocyte precursor cells (OPCs) to differentiate at the sites of injury. Thus, remission in MS is largely dependent upon OPCs migrating to sites of injury, and subsequently differentiating to a mature cell fate capable of repair (J. R. Patel, R. S. Klein, FEBS Lett, 585, 3730 (2011); D. Kremer et al, Ann Neurol, 69, 602 (2011); A. Chang et al, N Engl J Med, 346, 165 (2002)). Studies aimed at evaluating the presence and relative densities of OPCs at sites of chronically demyelinated MS lesions indicate that it is not a failure of repopulation or migration of OPCs, but rather inhibition of OPC differentiation at sites of injury that contributes to disease progression (D. M. Chari, W. F. Blakemore, Glia, 37, 307 (2002); D. M. Chari et al, J Neurosci Res, 73, 787 (2003); G. Wolswijk, J Neurosci, 18, 601 (1998); A. Chang et al, N Engl J Med, 346, 165 (2002); T. Kuhlmann et al, Brain, 131, 1749 (2008)).

There is no known cure for MS. For treating acute inflammatory attacks, intravenous corticosteroids are typically administered. Other treatments for MS involve the administration of an immunomodulator. Although immunomodulators are able to reduce the frequency and severity of attacks or accumulation of lesions, they do not promote remyelination of damaged axons.

Thus, there persists an unmet need for treatments that stimulate remyelination of axons and/or preserve myelination of axons in patients having multiple sclerosis (MS).

### BRIEF SUMMARY OF THE INVENTION

In some aspects, the present disclosure is directed to methods of preserving myelination of axons in a human subject having a demyelinating disease, comprising administering to the subject in need thereof an effective amount of a monoselective S1P receptor modulator.

In other aspects, the present disclosure is directed to a monoselective S1P receptor modulator for use in a method of preserving myelination of axons in a human subject having a demyelinating disease, comprising administering to the subject in need thereof an effective amount of the monoselective S1P receptor modulator.

In other aspects, the present disclosure is directed to the use of a monoselective S1P receptor modulator in the preparation of a medicament for preserving myelination of axons in a human subject having a demyelinating disease, wherein said medicament is adapted to administer to the subject in need thereof an effective amount of the monoselective S1P receptor modulator.

In other aspects, the present disclosure is directed to pharmaceutical products comprising a monoselective S1P receptor modulator, including kits. Typically, the kit comprises the pharmaceutical product and includes instructions for administering the monoselective S1P receptor modulator to a human subject having MS.

In some aspects, the monoselective S1P receptor modulator is an S1P1 monoselective receptor modulator. In some aspects, the S1P1 monoselective receptor modulator is ponesimod.

In some aspects, the demyelinating disease is multiple sclerosis. In some aspects, the multiple sclerosis is relapsing multiple sclerosis. In some aspects, the relapsing multiple sclerosis comprises relapsing-remitting disease, clinically isolated syndrome, or active secondary progressive disease.

### BRIEF DESCRIPTION OF THE DRAWINGS

FIG. 1 depicts results of an agarose drop migration assay used to determine alteration of primary OPC migration for ponesimod, A97, a combination of ponesimod and A97, and fingolimod phosphate as a positive control.

FIGS. 2A and 2B depict results of a differentiation assay used to determine if ponesimod, A97 and fingolimod phosphate affect primary mouse OPC differentiation.

FIG. 3 depicts results of immunohistochemical staining for myelin basic protein (MBP or Mbp) on primary mouse OPCs treated with ponesimod, A97, fingolimod phosphate, and 0.1% dimethylsulfoxide (DMSO) as a negative control.

FIGS. 4A-4H depict results of quantitative PCR used to determine if ponesimod and A97 affected primary mouse OPC differentiation.

### DETAILED DESCRIPTION OF ILLUSTRATIVE EMBODIMENTS

In the present disclosure the singular forms “a”, “an,” and “the” include the plural reference, and reference to a particular numerical value includes at least that particular value, unless the context clearly indicates otherwise. Thus, for example, a reference to “a material” is a reference to at least one of such materials and equivalents thereof known to those skilled in the art, and so forth.

When a value is expressed as an approximation by use of the descriptor “about,” it will be understood that the particular value forms another embodiment. In general, use of the term “about” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about”. In other cases, the gradations used in a series of values may be used to determine the intended range available to the term “about” for each value. Where present, all ranges are inclusive and combinable. That is, references to values stated in ranges include every value within that range.

When a list is presented, unless stated otherwise, it is to be understood that each individual element of that list and every combination of that list is to be interpreted as a separate embodiment. For example, a list of embodiments presented as “A, B, or C” is to be interpreted as including the embodiments, “A,” “B,” “C,” “A or B,” “A or C,” “B or C,” or “A, B, or C.”

It is to be appreciated that certain features of the disclosure which are, for clarity, described herein in the context of separate embodiments, may also be provided in combination in a single embodiment. That is, unless obviously incompatible or excluded, each individual embodiment is deemed to be combinable with any other embodiments and such a combination is considered to be another embodiment. Conversely, various features of the disclosure that are, for brevity, described in the context of a single embodiment, may also be provided separately or in any sub-combination. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation. Finally, while an embodiment may be described as part of a series of steps or part of a more general structure, each said step may also be considered an independent embodiment in itself.

In some aspects, the present disclosure is directed to methods of preserving myelination of axons in a human subject having a demyelinating disease, comprising administering to the subject in need thereof an effective amount of a monoselective S1P receptor modulator.

In some aspects, the present disclosure is directed to a monoselective S1P receptor modulator for use in a method of preserving myelination of axons in a human subject having a demyelinating disease, comprising administering to the subject in need thereof an effective amount of the monoselective S1P receptor modulator.

In some aspects, the present disclosure is directed to the use of a monoselective S1P receptor modulator in the preparation of a medicament for preserving myelination of axons in a human subject having a demyelinating disease, wherein said medicament is adapted to administer to the subject in need thereof an effective amount of the monoselective S1P receptor modulator.

In some aspects of the present disclosure, the monoselective S1P receptor modulator is administered in the absence of a non-selective S1P receptor modulator or another monoselective S1P receptor modulator. In some aspects of the present disclosure, the monoselective S1P receptor modulator is administered in the absence of a non-selective S1P receptor modulator. In some aspects of the present disclosure, the monoselective S1P receptor modulator is administered in the absence of another monoselective S1P receptor modulator. In some aspects of the present disclosure, the monoselective S1P receptor modulator is administered as a monotherapy.

In some aspects of the present disclosure, the monoselective S1P receptor modulator is an S1P1 monoselective receptor modulator. In some aspects of the present disclosure, the S1P1 monoselective receptor modulator is ponesimod. In some aspects, another monoselective S1P receptor modulator is a S1P5 monoselective receptor modulator.

As used herein, the term “monoselective S1P receptor modulator” (could also be referred to as “monospecific S1P receptor modulator”) refers to a S1P inhibitor small molecule that demonstrates a preference to predominantly bind to one of the five subtypes of S1P receptors (e.g., S1P1, S1P2, S1P3, S1P4 and S1P5). As used herein, the term “S1P1 monoselective receptor modulator” (could also be referred to as “S1P1 monospecific receptor modulator”) refers to a small molecule inhibitor that demonstrates a preference to predominantly bind to the S1P1 receptor over the other S1P receptors (e.g., S1P2, S1P3, S1P4 and S1P5).

As noted herein, A971432 (CAS no. 1240308-45-5), is an example of a monoselective S1P5 receptor modulator. In addition to ponesimod, other examples of monoselective S1P1 receptor modulators include AUY954 (CAS no. 820240-77-5), CS-0777 (CAS no. 840523-39-9), KRP-203 (CAS no. 509088-69-1), and SEW2871 (CAS no. 256414-75-2).

In some aspects of the present disclosure, the demyelinating disease is multiple sclerosis, idiopathic inflammatory demyelinating disease, transverse myelitis, Devic's disease, progressive multifocal leukoencephalopathy, optic neuritis, leukodystrophy, Guillain-Barre syndrome, chronic inflammatory demyelinating polyneuropathy, autoimmune peripheral neuropathy, Charcot-Marie-Tooth disease, acute disseminated encephalomyelitis, adrenoleukodystrophy, adrenomyeloneuropathy, Leber's hereditary optic neuropathy, or HTLV-associated myelopathy.

In some aspects of the present disclosure, the demyelinating disease is multiple sclerosis. In some aspects of the present disclosure, the multiple sclerosis is relapsing multiple sclerosis. In some aspects of the present disclosure, the relapsing multiple sclerosis comprises relapsing-remitting disease, clinically isolated syndrome, or active secondary progressive disease. In some aspects of the present disclosure, the relapsing multiple sclerosis comprises active secondary progressive disease.

In some aspects, the present disclosure is directed to methods of preserving myelination of axons in a human subject having multiple sclerosis, comprising administering to the subject in need thereof an effective amount of ponesimod.

As used herein, the term “preserving myelination of axons” refers to preventing the patient's axon myelination levels from decreasing relative to the patient's axon myelination levels at baseline, wherein baseline refers to a time period prior to initiation of treatment with ponesimod. This time period is typically up to about 45 days prior to initiation of treatment with ponesimod, including, for example, up to about 40 days, up to about 35 days, up to about 30 days, up to about 25 days, up to about 20 days, up to about 15 days, or up to about 10 days prior to initiation of treatment with ponesimod. By preserving myelination, the methods otherwise relate to stabilizing axon myelination and/or stimulating axon remyelination. In certain embodiments, a limited amount of decrease to a patient's axon myelination levels may occur without deviating from the preservation effects reflected herein.

As demonstrated herein (see Example 1), an S1P1 receptor modulator alone has been found not to inhibit OPC migration whereas the dual modulation of S1P1 and S1P5 receptors (by a non-selective S1P receptor modulator or by a combination of monoselective receptor modulators) have been found to lead to inhibition of OPC migration. Insufficient OPC migration into demyelinated lesions has been found to be a cause of poor remyelination in MS. In addition, the methods described herein also lead to enhanced OPC differentiation. As demonstrated in Example 1, enhanced OPC differentiation in response to high concentrations of ponesimod (300 nM-1000 nM) was observed, as well as modest increases in response to high concentration (1000 nM) of A971432, a monoselective S1P5 receptor modulator.

In some aspects of the present disclosure, the methods are performed on a human subject having multiple sclerosis. In some embodiments, the subject's multiple sclerosis is relapsing multiple sclerosis. In other embodiments, the relapsing multiple sclerosis comprises relapsing-remitting disease, clinically isolated syndrome, or active secondary progressive disease.

In some aspects of the methods of the present disclosure, the human subject is administered an effective regimen of ponesimod. An effective regimen is one that elicits the biological or medicinal response in a human tissue system that is being sought by a researcher, medical doctor, or other clinician, which includes preservation of myelination of axons. In some embodiments, the human subject is treatment naïve prior to being administered an effective regimen of ponesimod.

In some embodiments, the human subject is at an early stage of disease progression and/or has experienced limited demyelination of axons. In some embodiments, the human subject is at an early stage of disease progression. In some embodiments, the human subject has experienced limited demyelination of axons.

As used herein, the term “early stage of disease progression” refers to a stage of the disease that results in the patient or patient population having a baseline expanded disability status scale (EDSS) score of ≤3 and optionally wherein the patient or patient population is/are also treatment naïve or undergoing a first platform switch, e.g., a switch from low efficacy disease modifying therapy with a low risk tolerance. These include, for example, older injectable (intramuscular and subcutaneous) forms of DMT for MS such as recombinant human interferon beta-1b, recombinant human interferon beta-1a, and glatiramer acetate.

In some embodiments, the methods are directed to a patient or patient population that is/are at an early stage of disease progression and/or with limited demyelination of axons. In some embodiments, the patient or patient population that is/are at an early stage of disease progression and/or have limited demyelination of axons have a baseline expanded disability status scale (EDSS) score of ≤3. In some embodiments, the patient or patient population that is/are at an early stage of disease progression and/or have limited demyelination of axons have a baseline expanded disability status scale (EDSS) score of ≤3 and the patient or patient population is/are also treatment naive. In some embodiments, the patient or patient population that is/are at an early stage of disease progression and/or have limited demyelination of axons have a baseline expanded disability status scale (EDSS) score of ≤3 and the patient or patient population is/are undergoing a first platform switch.

As used herein, the term “ponesimod” refers to the compound (R)-5-[3-chloro-4-(2,3-dihydroxy-propoxy)-benz[Z]ylidene]-2-([Z]-propylimino)-3-o-tolyl-thiazolidin-4-one, which has the following structure (see also RE43,728, incorporated herein by reference):

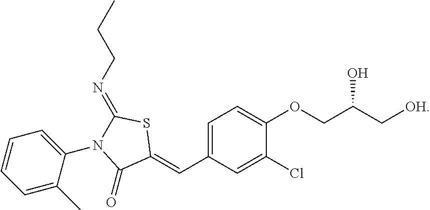 [SMILES: CCC/N=C1\S/C(=C\c2ccc(OC[C@H](O)CO)c(Cl)c2)C(=O)N1c1ccccc1C]

“Ponesimod” also refers to pharmaceutically acceptable salts of ponesimod. The term “pharmaceutically acceptable salt” refers to salts that retain the desired biological activity of the subject compound and exhibit minimal undesired toxicological effects. Such salts include inorganic or organic acid and/or base addition salts depending on the presence of basic and/or acidic groups in the subject compound. For reference see for example Handbook of Pharmaceutical Salts. Properties, Selection and Use, P. Heinrich Stahl, Camille G. Wermuth (Eds.), Wiley-VCH, 2008 and Pharmaceutical Salts and Co-crystals, Johan Wouters and Luc Quéré (Eds.), RSC Publishing, 2012.

It is to be understood that the present disclosure encompasses ponesimod in any form including amorphous as well as crystalline forms. It is further to be understood that crystalline forms of ponesimod encompasses all types of crystalline forms including polymorphs, solvates and hydrates, salts and co-crystals (when the same molecule can be co-crystallized with different co-crystal formers) provided they are suitable for pharmaceutical administration. In some embodiments, ponesimod is in crystalline form A or crystalline form C as described in WO 2010/046835, incorporated herein by reference. In some embodiments, ponesimod is in crystalline form C.

It should be noted that the amounts of ponesimod described herein are set forth on a ponesimod free base basis. That is, the amounts indicate that amount of the ponesimod molecule administered, exclusive of, for example, solvent (such as in solvates) or counterions (such as in pharmaceutically acceptable salts).

In some embodiments, the effective regimen comprises a daily dose of ponesimod. In some embodiments, the daily dose of ponesimod is administered orally. In some embodiments, the daily dose of ponesimod is administered once daily.

In some embodiments, the daily dose of ponesimod is about 2 mg. In some embodiments, the daily dose of ponesimod is about 3 mg. In some embodiments, the daily dose of ponesimod is about 4 mg. In some embodiments, the daily dose of ponesimod is about 5 mg. In some embodiments, the daily dose of ponesimod is about 6 mg. In some embodiments, the daily dose of ponesimod is about 7 mg. In some embodiments, the daily dose of ponesimod is about 8 mg. In some embodiments, the daily dose of ponesimod is about 9 mg. In some embodiments, the daily dose of ponesimod is about 10 mg. In some embodiments, the daily dose of ponesimod is about 20 mg.

In some embodiments, about 20 mg of ponesimod is administered orally once daily.

In other embodiments, the effective regimen comprises an up-titration, followed by a daily maintenance dose of ponesimod. An up-titration is a dosing procedure in which the daily dose of ponesimod is gradually increased over a period of days, culminating with administration of the maintenance dose. An exemplary up-titration regimen is disclosed in U.S. Pat. No. 10,220,023, incorporated herein by reference.

In some embodiments, the regimen comprises an up-titration at the initiation of the method of the disclosure. In other embodiments, the regimen comprises an up-titration upon re-initiation of the method after a discontinuation of the method of the disclosure. As used herein, “upon re-initiation of the method after a discontinuation” means an interruption of the administration of ponesimod of at least one, at least two or preferably at least 3 days before treatment is re-initiated. In some embodiments, the regimen comprises an up-titration step at initiation of the method or upon re-initiation of the method after a discontinuation.

In other embodiments of the methods of the disclosure, the up-titration comprises administering orally once daily about 2 mg of ponesimod on days 1 and 2; about 3 mg of ponesimod on days 3 and 4; about 4 mg of ponesimod on days 5 and 6; about 5 mg of ponesimod on day 7; about 6 mg of ponesimod on day 8; about 7 mg of ponesimod on day 9; about 8 mg of ponesimod on day 10; about 9 mg of ponesimod on day 11; about 10 mg of ponesimod on days 12, 13 and 14; followed by administering a maintenance dose of about 20 mg of ponesimod once daily thereafter beginning on day 15.

In some embodiments, the maintenance dose is about 20 mg of ponesimod once daily.

The present disclosure also provides pharmaceutical products comprising ponesimod, including kits. Typically, the pharmaceutical product comprises a package, such as a bottle, a pouch, or a blister pack. Kits include can include, for example, the package, instructions sheets, or other components for use by the patient to facilitate administration.

In some embodiments, the package and/or kit includes instructions. In certain embodiments, instructions are for administering ponesimod to a human subject having MS in a regimen that is effective to preserve myelination of axons. In other embodiments, the package provides instructions directed to subjects who are treatment naïve. In other embodiments, the package provides instructions directed to subjects at an early stage of disease progression. In other embodiments, the package provides instructions directed to subjects having experienced limited demyelination of axons.

The following Example is provided to illustrate some of the concepts described within this disclosure. While the Example is considered to provide an embodiment, it should not be considered to limit the more general embodiments described herein.

### EXAMPLES

### Example 1

### Study Design

The effects of ponesimod, a selective S1P1 receptor agonist, and A971432, a selective S1P5 receptor agonist, on oligodendrocyte precursor cell (OPC) migration and differentiation were evaluated. Nine concentrations were tested (logarithmic dilutions ranging from 0.3 nM to 3000 nM, n=6 per concentration). Fingolimod phosphate (P-FTY720, 3000 nM, n=6) was included as a positive control, 0.1% DMSO was included (min.one/plate) as a negative control.

Primary mouse OPCs were harvested on postnatal day 0 or 1 and mixed glia cultures were maintained for 14 days. Primary OPCs were then purified using the shake-off method, and used for either a migration assay, or a differentiation assay, as detailed below.

### Methodology

### Animals

Thirty-five female and eleven male C57BL/6J breeding animals were purchased from Envigo (Venray, The Netherlands), and used to generate a total of 334 pups. Females were housed in pairs in standard enriched type III cages according to the ethical protocol (ID202049B), on a 12hr light-dark cycle (lights on at 6:30 am, lights off at 6:30 pm), in a temperature and humidity-controlled room. Food and water was available at libitum.

### Compounds

Ponesimod was provided by Janssen Pharmaceutics. FTY720 Phosphate was ordered from SANBIO bv (Uden, The Netherlands). A971432 (reported as A97) was ordered from Bio-Techne Ltd (Abingdon, UK), CAS number 1240308-45-5.

### Primary OPC Culture

To harvest primary mouse OPCs, cortices are isolated from mouse pups on postnatal day 0 or 1 and placed in ice-cold Dulbecco's Modified Eagle Medium (DMEM) (Sigma Aldrich, Overijse, BE). The cortices are homogenized and centrifuged for 5 minutes at 300 G. The supernatant is removed, and a papain solution is added for 30 minutes (125 uL DMEM solution per brain containing 20 units/ml papain and 40 μg/ml DNase, at 37 degrees). Digestion is inactivated by adding 9 ml cold DMEM. The solution is then centrifuged for 5 minutes at 300G. The supernatant is carefully removed, and the pellet is resuspended in 2-5 ml culture medium (DMEM with 10% fetal calf serum (Biowest, Nuaillé, FR) and 1% pen/strep (Sigma Aldrich, Overijse, BE)). Cells are triturated gently with a 21 g needle. Cell suspension is divided over PLL (Sigma Aldrich, Overijse, BE)-coated flasks (2 brains per flask) and DMEM is added to a final volume of 10 ml. DMEM is replaced on day 4, 7, and 11. On day 7 and 11, insulin (Sigma Aldrich, Overijse, BE) is added to the medium (5μg/ml).

On day 14, OPCs are separated from astrocytes by the shake-off method: flasks are placed on a shaker for 45 minutes at 75 rpm (37 degrees). The medium is then aspirated and discarded, and 10 ml fresh culture medium is added. Flasks are then returned to the shaker for 16-18 hours shaking at 280 rpm. Next, the medium is isolated, and this cell suspension is placed in a plastic petri dish and incubated for 25 minutes. The medium, which contains the OPCs, is then collected and centrifuged for 5 minutes at 300 G. Cells are then resuspended in SATO differentiation medium containing 2% horse serum, 2% B-27, 50 U/ml P/S, 2.4 μl/ml transferrin, 1.04 μl/ml L-thyroxine, 1 μl/ml putrescin, 1 μl/ml progesterone, 1 μl/ml TIT, 0.74 μl/ml sodium selenite and 0.5 μl/ml insulin (all from Sigma-Aldrich, Overijse, BE). The cells are triturated, counted and plated onto 24-well plates.

### Migration Assay

After shake-off, 20 ul of 1% agarose is added to 40 ul cell suspension (cell density between 20 and 100×106 cells/ml). Drops of 1.5 uL cell suspension are dropped at the centre of wells in a 24-well PLL-coated tissue culture plate. After setting for 15 minutes at 4 degrees Celsius, 50 uL SATO's medium is added around the drop. After 2 hours, 450 ul SATO's medium is added. Test or control compound is added to the medium. The extent of cell migration is then measured after 5 days using Incucyte imaging analysis and fluorescent endpoint staining. For each well, the 15-20 cells that migrated the furthest away from the agarose drop are measured and averaged.

### Differentiation Assay

After shake-off, cells are reset for their differentiation status: one hour after plating, cells are treated with platelet-derived growth factor (PDGF, 10 ng/ml, Peprotech EC Ltd, London, UK) and fibroblast growth factor (fgf, 10 ng/ml, Peprotech EC Ltd, London, UK). Twenty-four hours after plating, cells are treated again with fgf (10 ng/ml).

### Immunocytochemistry

150,000 cells are plated onto glass coverslips in 24-well plates. Cells are treated with test compound, control compound or DMSO on day 1 (twenty-four hours after fgf treatment), day 3 and day 5, and fixated in 4% paraformaldehyde for 30 minutes at room temperature. Primary OPCs are fixed in 4% paraformaldehyde (PFA) for 30 minutes at room temperature. Aspecific binding was blocked for 30 minutes with 1% bovine serum albumin (BSA) in 0.1% PBS-T, followed by incubation with primary antibodies (O4:R&D systems, Minneapolis, USA, MBP:Merck, Overijse, BE) for four hours at room temperature. After three washing steps with PBS (Lonza group, Bornem, BE), cells are incubated with Invitrogen Alexa Fluor 488 (Alexa 488) or Invitrogen Alexa Fluor 555 (Alexa 555) conjugated secondary antibody for one hour (Life technologies, Carlsbad, USA). Nuclei are counterstained with 4′6-diamidino-2-phenylindole (DAPI; Sigma-Aldrich, Overijse, USA). Coverslips are mounted with Dako mounting medium (Dako, Carpinteria, USA) and analyzed using a fluorescence microscope (Leica DM2000 LED). Images are quantified using Fiji, ImageJ software.

Quantitative PCR (qPCR)

250,000 cells are plated onto glass coverslips in 24-well plates. Cells are treated with test compound or DMSO on day 1 (twenty-four hours after fgf treatment) and 3, and lysated on day 4. Total RNA was isolated from primary OPCs, using the RNeasy mini kit (Qiagen, Venlo, the Netherlands), according to the manufacturer's instructions. RNA concentration and quality is analyzed with a Nanodrop spectrophotometer (Isogen Life Science, Leiden, The Netherlands). RNA is reverse-transcribed using the qScript cDNA Supermix kit (Quanta, Leuven, Belgium). qPCR is performed to analyze gene expression, using the Applied Biosystems QuantStudio 3 Real-Time PCR System (Life Technologies, Gent, Belgium). The reaction mixture consists of SYBR Green master mix (Life Technologies, Carlsbad, USA), 10 μM forward and reverse primers (Integrated DNA Technologies, Leuven, Belgium), nuclease-free water and cDNA template (12.5 ng), up to a total reaction volume of 10 μl. Results are analyzed by the comparative Ct method and normalized to the most stable housekeeping genes (β-actin, Ywhaz), determined by GeNorm.

| Primer | | |
|---|---|---|
| list | Forward (5′-3′) | Reverse (5′-3′) |
| Ywhaz | GCAACGATGTACGTCTCTTTTGG | GTACACAATTCCTTTCTTGTCATC |
| B-actin | GGCTGTATTCCCCTCCATCG | CAGTTGGTAACAATGCCATGT |
| Mbp | TCACAGAAGAGACCCTCACAGC | GAGTCAAGGATGCCCGTGTC |
| Plp | TTGTTTGGGAAAATGGCTAGGA | GCAGATGGACAGAAGGTTGGA |
| Mog | TCCGTGCAGAAGTCGAGAA | CCATCACTCAAAAGGGGTTTC |
| Mag | ACACCCCCAACATTGTGGTT | CGAACTGCAAGGTGGTGTTG |
| Mobp | TACTCGCAGGGCAACAAAGA | CACTGGGTTTTCATCCGTGC |
| Pdgfra | TCCTTCTACCACCTCAGCGAG | CCGGATGGTCACTCTTTAGGAAG |
| Ng2 | GGATCGCCAGAGCCAGCCA | GGGCACAATAGCCCCCTCCA |

Ywhaz = Tyrosine 3-Monooxygenase/Tryptophan 5-Monooxygenase Activation Protein Zeta; Mbp = myelin basic protein; Plp = proteolipid protein; Mog = myelin oligodendrocyte glycoprotein; Mag = myelin associated glycoprotein; Mobp = myelin associated oligodendrocyte basic protein; Pdgfra = platelet-derived growth factor receptor alpha; Ng2 = neuron-glial antigen 2

### Results

### Migration Assay

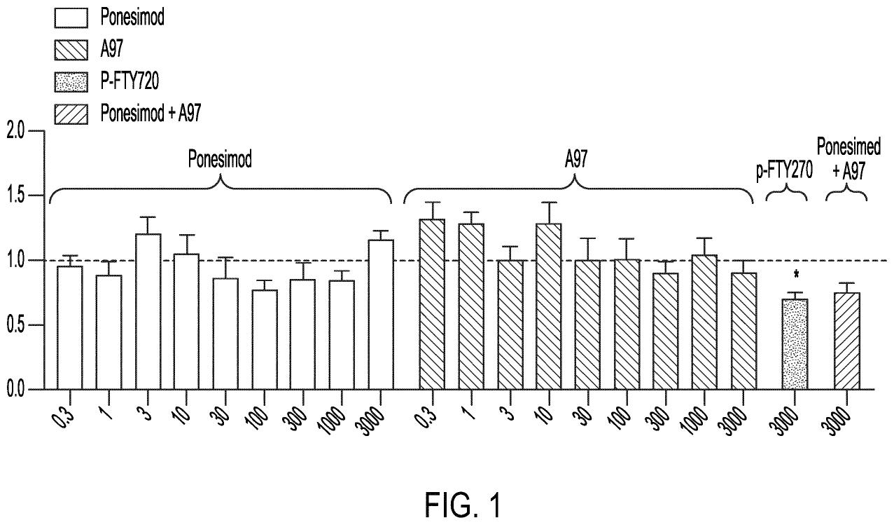

Ponesimod and A97 effect on migration of primary mouse OPCs using an agarose drop migration assay was investigated. Fingolimod phosphate was included as a positive control, 0.1% DMSO was included as a negative control against which the data was normalized (i.e., within plate). One sample t-tests were performed to detect increase/decrease in migration, and significance level was corrected for multiple comparisons (α/20 groups=0.0025). One sample t-tests revealed significantly decreased migration distance when OPCs were treated 3000 nM fingolimod phosphate (t6=6.454, p=0.0007), as shown in FIG. 1.

Ponesimod and A97 did not affect migration distance at any of the concentrations tested. The combined treatment with A97 and ponesimod 3000 nM did not significantly alter migration either, after correction for alpha (t4=3.597, p=0.023), but it should be noted that this is a smaller sample size. As seen in FIG. 1, while not statistically significant, combined treatment with ponesimod and A97 demonstrated a trend towards inhibited migration. Positive control fingolimod phosphate significantly inhibited primary OPC migration. *p<0.0025.

### Differentiation Assay

### Immunocytochemistry

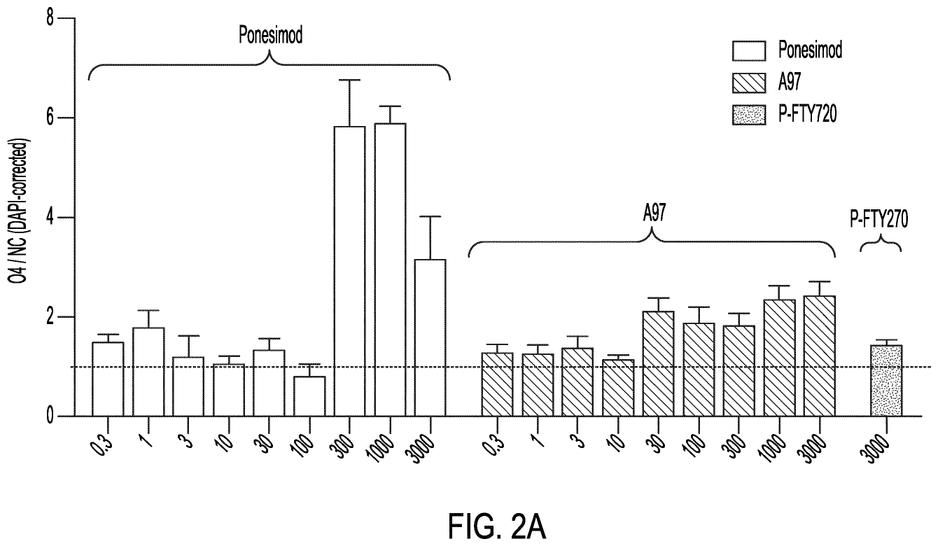

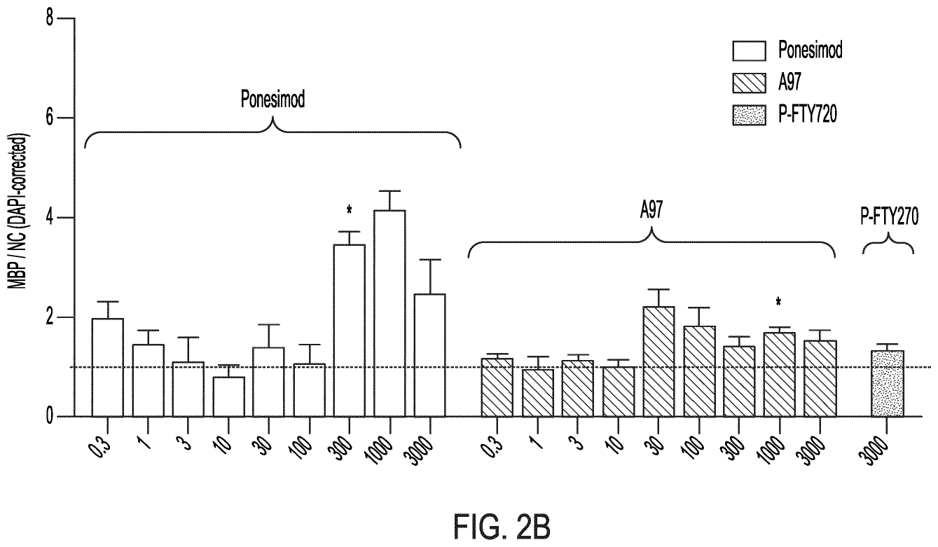

Ponesimod and A97 effect on primary mouse OPC differentiation using immunocytochemical staining for O4 and MBP, markers of early and late differentiation stages, respectively, was measured. Fingolimod phosphate, which served as a positive control in the migration assay, was included as well, 0.1% DMSO was included as a negative control. All data were normalized to DAPI (i.e., within well) and negative controls (i.e., within plate): compound O4 or MBP/DAPI normalized to NC O4 or MBP/DAPI on the same plate and the results are shown in FIGS. 2A and 2B.

One sample t-tests were performed, and alpha was corrected for multiple comparisons (α/19 comparisons=0.0026). Cells from one plate needed to be excluded from analyses due to aberrant O4 and MBP to DAPI ratios, identified using the ROUT method for identifying outliers (Graphpad Prism). Immunohistochemical staining for O4 (FIG. 2A) and MBP (FIG. 2B) revealed marginally enhanced O4 expression in response to 1000 nM ponesimod as well as 1000 nM and 3000 nM A97. Moreover, MBP expression was significantly increased in response to 300 nM ponesimod and 1000 nM A97. *p<0.0025.

Fluorescent immunocytochemistry shows marginally increased O4 expression in primary OPCs that had been treated with 1000 nM ponesimod (t2=16.393, p=0.0037), as well as 1000 nM A97 (t5=5.158, p=0.0036) and 3000 nM A97 (t5=5.214, p=0.0034).

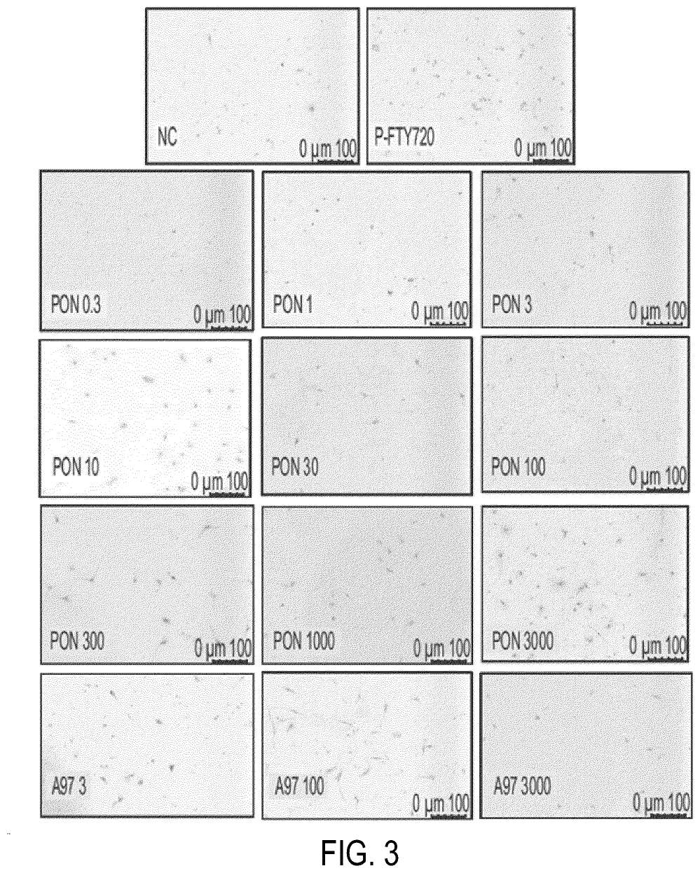

A significant increase in MBP expression in primary OPCs that had been treated with 300 nM ponesimod (t3=10.842, p=0.0017) and 1000 nM A97 (t5=8.153, p=0.0005) was observed, as shown in FIG. 3. 300 nM ponesimod did not show significantly increased O4 expression (t3=5.393, p=0.012) after correction for significance level, yet this apparent lack of effect might be due to a smaller sample size (as a result of exclusion of data). Similarly, 1000 nM ponesimod did not show significantly increased MBP expression after correction for significance level (t2=2.148, p=0.012), again possibly due to the smaller sample size.

### Quantitative PCR

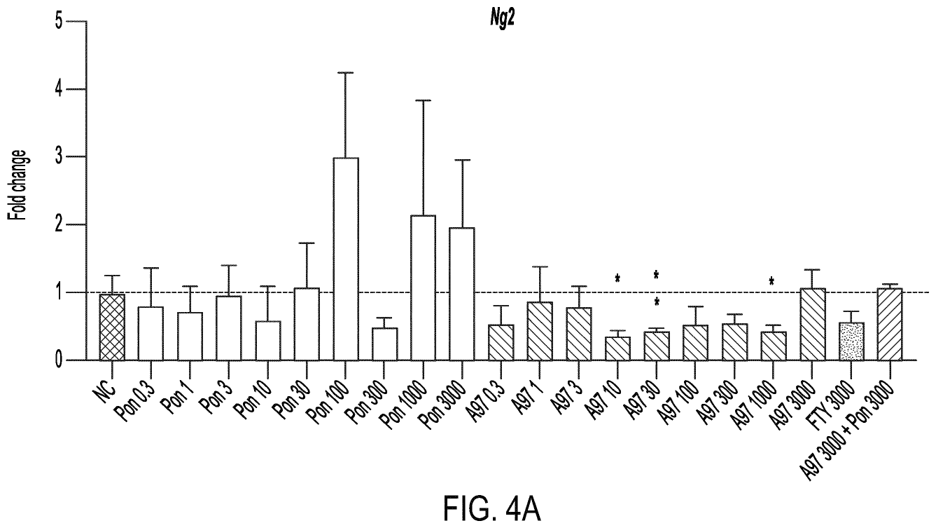

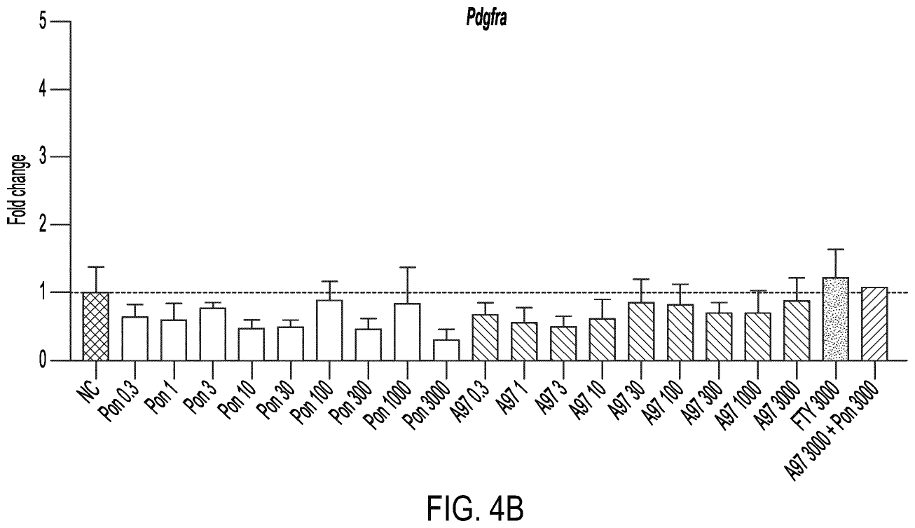

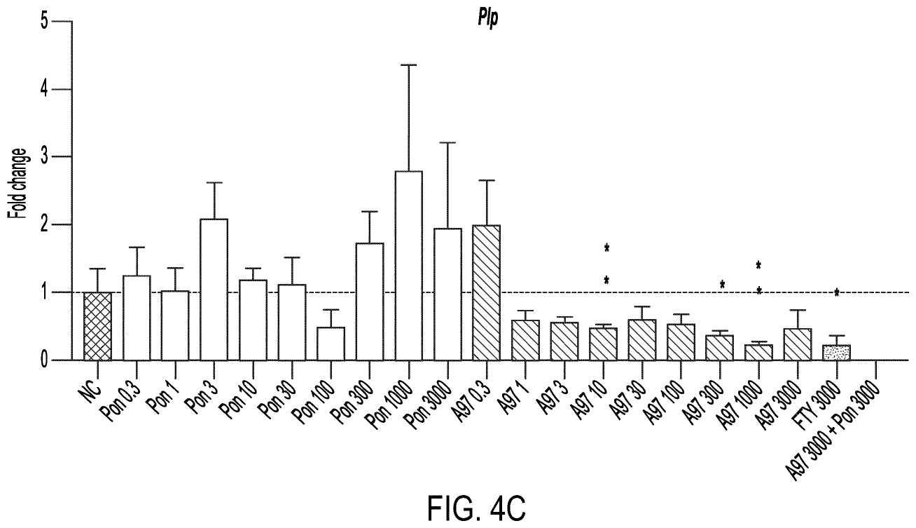

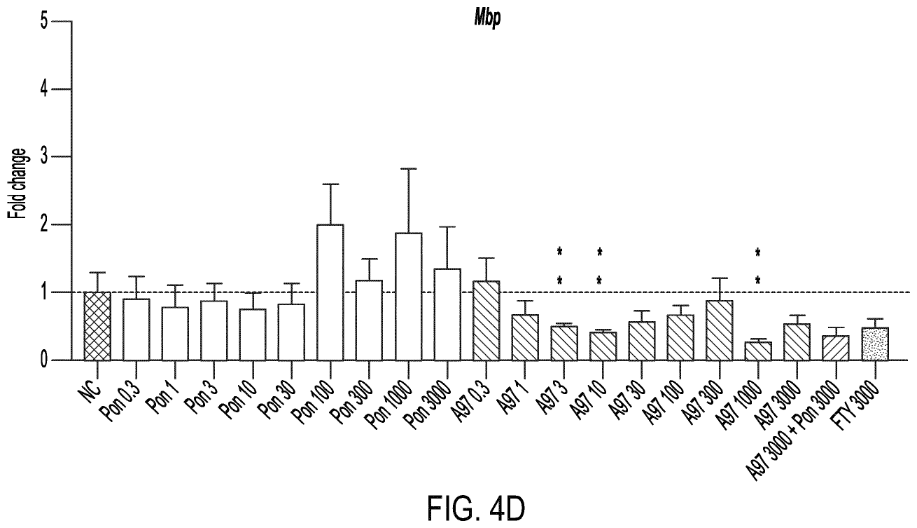

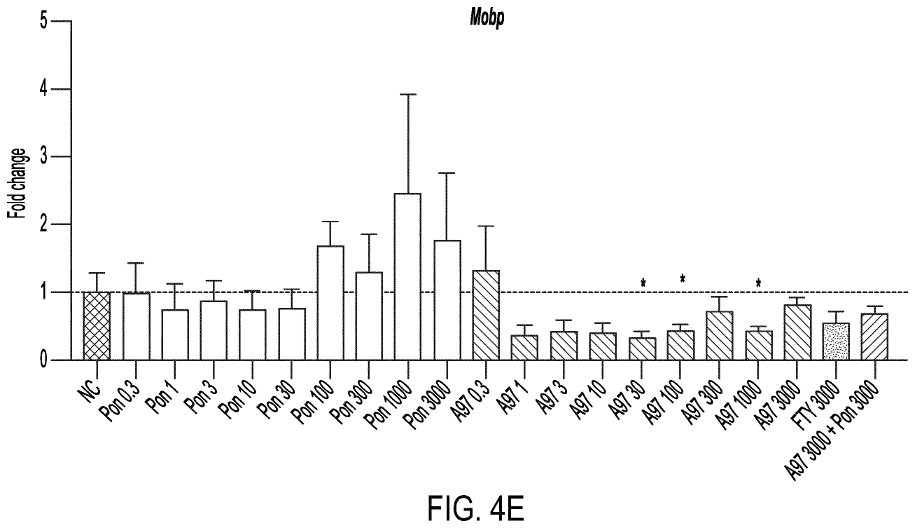

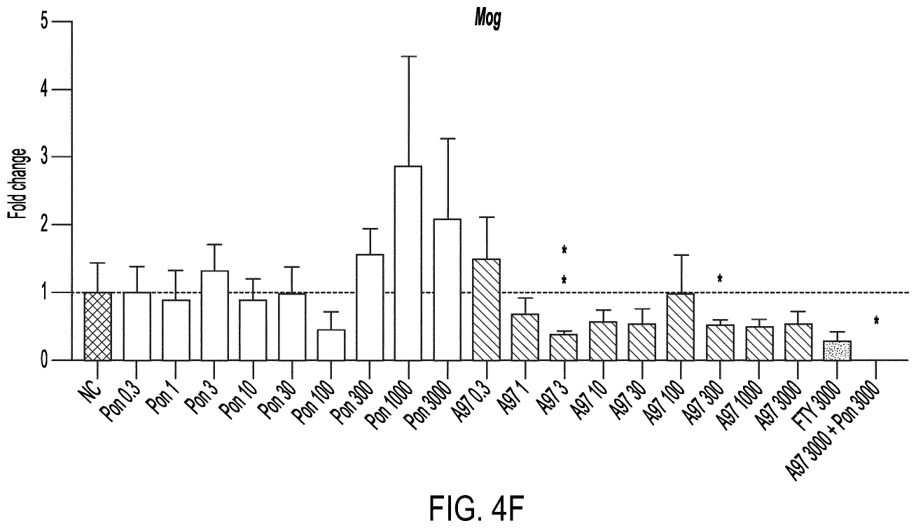

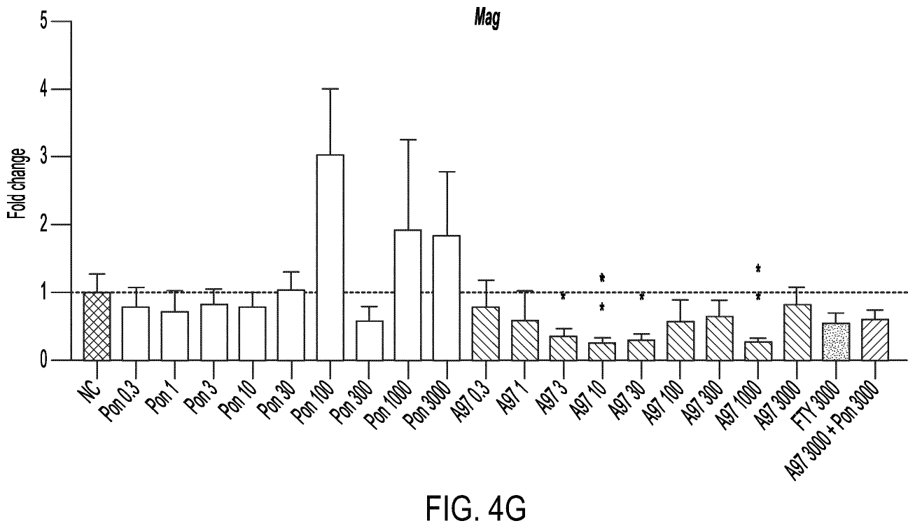

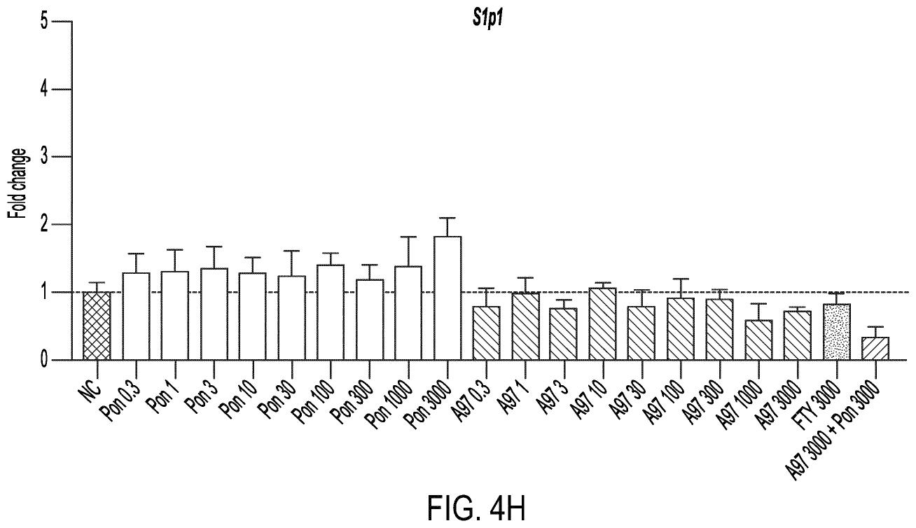

Ponesimod and A971432 effect on primary mouse OPC differentiation using qPCR was measured and the results are shown in FIGS. 4A-4H. All data was normalised against plate-matched negative controls. One sample t-tests were performed and corrected for multiple testing (α/20 comparisons=0.0025). *p<0.0025, **p<0.0005.

No significant changes at the gene expression level were observed when primary OPCs are treated with ponesimod. However, treatment with A97 induces significant concentration-dependent decreases in expression of Ng2, Plp, Mbp, Mobp, Mog, and Mag.

A decrease in Mbp expression in response to 3 nM A97 (t5=11, p<0.0001), 10 nM A97 (t5=19, p<0.0001) and 1000 nM A97 (t5=15, p<0.0001) was observed (see FIG. 4D).

For PLP, a significant decrease in expression in response to 10 nM A97 (t5=9.941, p=0.0002), 300 nM A97 (t5=7.764, p=0.0006), 1000 nM A97 (t5=15.05, p=0.0001) and combined 3000 nM A97 and 3000 nM ponesimod (t1=545.7, p=0.0012) was observed (see FIG. 4C).

For MOG, a significant decrease in expression in response to 3 nM A97 (t5=10.92, p=0.0001), 300 nM A97 (t5=6.037, p=0.0018) and combined 300 nM ponesimod with 300 nM A97 (t1=396, p=0.0016) was observed (see FIG. 4F).

For Mobp, a significant decrease in expression for 30 nM A97 (t5=6.67, p=0.0011), 100 nM A97 (t5=6.025, p=0.0018), and 1000 nM A97 (t5=7.939, p=0.0005) was observed (see FIG. 4E).

For Mag, a significant decrease in gene expression in response to 3 nM A97 (t5=5.97, p=0.0019), 10 nM A97 (t5=9.229, p=0.0003), 30 nM A97 (t5=7.8883, p=0.0005), and 1000 nM A97 (t5=13.82, p<0.0001) was observed (see FIG. 4G).

For Ng2, a significant decrease in gene expression in response to 10 nM A97 (t5=6.071, p=0.0018), 30 nM A97 (t5=8.706, p=0.0003), and 1000 nM A97 (t5=6.072, p=0.0017) was observed (see FIG. 4A).

For PDGFRa and S1P1, no significant change were observed (see FIGS. 4B and 4H, respectively).

### Conclusion

The effects of ponesimod, a selective S1P1 receptor agonist, and A971432, a selective S1P5 receptor agonist, on oligodendrocyte precursor cell (OPC) migration and differentiation were evaluated. No effects of S1P1 or S1P5 separately on primary mouse OPC migration were observed. In contrast, fingolimod phosphate does inhibit primary mouse OPC migration. Of note, combined treatment with ponesimod and A97 revealed a trend towards inhibited migration as well.

In addition, alteration of differentiation of primary mouse OPCs was evaluated by activating the S1P1 receptor or the S1P5 receptor. Enhanced OPC differentiation in response to high concentrations of ponesimod (300 nM -1000 nM) was observed, as well as modest increases in response to high concentration (1000 nM) of A97, revealed by fluorescent immunohistochemical staining for O4 and MBP. Quantitative PCR data showed more variable results, possibly due to the multiple correction in the analysis being a snapshot in time early on in the differentiation process. The non-significant trends in gene expression for myelin genes predict differentiation seen in intraclass correlation coefficient (ICC) analyses.

### Example 2

### Study Design

A prospective, multicenter, randomized, double-blind, active controlled, parallel-group, phase III, superiority study (Phase III OPTIMUM study) was conducted. The study was designed to compare the efficacy, safety, and tolerability of ponesimod 20 mg vs teriflunomide 14 mg in adult subjects with relapsing MS.

Randomization: Subjects were randomized in a 1:1 ratio to ponesimod 20 mg or teriflunomide 14 mg, stratified by prior use of MS disease modifying treatment (DMT) in the last two years prior to randomization (yes, no) and by baseline expanded disability status scale (EDSS) score (EDSS≤3.5, EDSS>3.5).

### Inclusion Criteria

This study enrolled adult male and female subjects aged 18 to 55 years with established diagnosis of MS, as defined by the 2010 revision of McDonald Diagnostic Criteria [Polman C H, et al. Diagnostic criteria for multiple sclerosis: 2010 revisions to the McDonald criteria. Ann Neurol. 2011; 69(2):292-302], with relapsing course from onset (i.e., relapsing-remitting multiple sclerosis and secondary progressive multiple sclerosis [SPMS] with superimposed relapses). The trial included up to a maximum 15% of subjects with SPMS with superimposed relapses.

Subjects had active disease evidenced by one or more MS attacks with onset within the period of 12 to 1 months prior to baseline EDSS assessment, or by two or more MS attacks with onset within the 24 to 1 months prior to baseline EDSS assessment, or with one or more gadolinium-enhancing (Gd+) lesion(s) of the brain on an MRI performed within 6 months prior to baseline EDSS assessment. Enrolled subjects were ambulatory with an EDSS score of up to 5.5 inclusive. The subjects were treatment-naïve (i.e., no MS disease-modifying therapy received at any time in the past) or previously treated with interferon (IFN) β-1a, IFN β-1b, glatiramer acetate, dimethyl fumarate, or natalizumab.

### Exclusion Criteria:

Subjects with significant medical conditions or therapies for such conditions (e.g., cardiovascular, pulmonary, immunological, hepatic, ophthalmological, ocular) or lactating or pregnant women were not eligible to enter the study.

Subjects with contraindications to MRI or with clinically relevant medical or surgical conditions that, in the opinion of the investigator, would put the subject at risk by participating in the study were not eligible to enter the study.

### Study/Treatment Duration:

For an individual subject, the maximum duration of the study was approximately 118 weeks consisting of 6 weeks of screening, 108 weeks of treatment and 4 weeks of safety follow-up. Subjects discontinuing treatment prematurely had an option to stay in a post-treatment observation period (PTOP) for up to 108 weeks.

### The Study Consisted of the Following Periods:

Pre-randomization period—Up to 45 days before randomization.
Treatment period: The double-blind treatment period lasted for 108 weeks. It consisted of a randomization visit, visits at two, four, and 12 weeks after randomization, and 12-weekly visits thereafter.

### End-of-Treatment (EOT):

The EOT visit took place at Week 108 (or earlier in case of premature discontinuation of study drug). In all cases, the EOT visit took place one day after the last dose of study drug but no later than 7 days after the last dose of study drug.

Subjects who completed treatment until Week 108 were eligible to enroll in an extension study conducted under a separate protocol. Subjects who discontinued study drug prematurely for any reason were not eligible for the extension study.

Subjects who prematurely discontinued study drug treatment were subsequently treated according to local standard of care at the investigator's discretion and were followed in the post-treatment observation period.

### Post-Treatment Safety Follow-up (FU) Period:

Teriflunomide is eliminated slowly from plasma. An accelerated elimination procedure was used by all subjects after the last dose of study drug. A safety FU after the last dose of study drug was mandated.

### All Subjects Entered the Safety FU Period:

For subjects who entered the extension study, the FU period started after the last dose of study drug and ended with a safety FU visit (FU1) 14-22 days after the last dose of study drug or with an abbreviated FU2 23-37 days after the last dose of study drug (if compliance to the teriflunomide accelerated elimination procedure was assessed as not sufficient at FU1).

For subjects who did not enter the extension study, the safety FU period lasted for 30 days after the last dose of study drug and included two safety FU visits (FU1, FU2) at 14-22 and 30-37 days after the last dose of study drug, respectively.

### Post-Treatment Observation Period (PTOP):

Subjects who prematurely discontinued study treatment enter the PTOP which lasts until 108 weeks after randomization (i.e., planned EOT period). It consisted of an abbreviated schedule of assessments at the time of the originally scheduled 12-weekly visits.

### End-of-Study (EOS)

EOS was reached when treatment, safety FU, and, if applicable, PTOP have been completed.

For subjects who completed the 108-week treatment period and entered the extension study, the EOS visit corresponded to the FU visit (FU1) conducted 14-22 days after the last study drug dose or to the abbreviated FU2 visit conducted 23-37 days after the last study drug dose (if needed for compliance reasons with the teriflunomide accelerated elimination procedure).

For all other subjects, the EOS visit corresponded to the 30-day FU visit (FU2) or to the last visit of PTOP (i.e., Week 108 Visit of the PTOP), whichever was last.

### Study Treatment:

The treatment period consisted of an up-titration period (from Day 1 to 14) and a maintenance period (Day 15 until EOT).

During an initial phase of the study, the study drugs in the up-titration period were administered in a double-dummy fashion. Ponesimod (or matching placebo) was presented as tablet, and teriflunomide 14 mg (or matching placebo) was presented as capsule (i.e., daily administration of one tablet and one capsule). At a later phase, the double-dummy material (tablet and capsule) was replaced by the daily administration of one capsule containing either ponesimod or teriflunomide.

In the maintenance period, the study treatment consisted of the daily administration of one capsule containing ponesimod 20 mg or teriflunomide 14 mg.

To reduce the first-dose effect of ponesimod, an up-titration scheme was implemented from Day 1 to Day 14:
Days 1 and 2; 2 mg.
Days 3 and 4; 3 mg.
Days 5 and 6; 4 mg.
Day 7; 5 mg.
Day 8; 6 mg.
Day 9; 7 mg.
Day 10; 8 mg.
Day 11; 9 mg.
Days 12, 13, and 14; 10 mg.
Day 15 until EOT; 20 mg.

Main analysis set for efficacy: The Full Analysis Set (FAS) included all randomized subjects. Subjects were evaluated according to the treatment they were randomized to.

Efficacy variable/timepoint: The endpoint was increase in ventricular volume up to the end of study (EOS). All available data up to EOS, regardless of treatment discontinuation was included (ITT approach).

### Statistical Methods

The Full Analysis Set (FAS) included all randomized subjects. In order to adhere to the intention-to-treat principle as much as possible, subjects were evaluated according to the treatment they have been randomized to.

The Per-Protocol Set (PPS) comprises all subjects included in the FAS without any major protocol deviations, that impact the assessment of the endpoint, occurring prior to or at randomization.

The Safety Set (SAF) included all randomized subjects who received at least one dose of study treatment. Subjects were analyzed based on actual treatment taken, not randomized treatment.

### Disposition and Baseline Characteristics:

A total of 1133 subjects were randomized to the study, 567 to ponesimod 20 mg and 566 to teriflunomide 14 mg. Overall treatment and study discontinuation were balanced across both treatment arms, 83% of subjects completed treatment. The mean age was 36.7 years and 64.9% of subjects were female. Most subjects were recruited in Europe with 50.6% from EU countries. Mean baseline EDSS score was 2.6 and mean disease duration was 7.6 years. Mean pre-study 12-month relapse rate was 1.3, and 42.6% subjects had ≥1 gadolinium-enhancing (Gd+) Tl lesions. The treatment arms were generally balanced in terms of demographics and baseline disease characteristics.

### 1. Subject And Treatment Information

A total of 1468 subjects were screened. Of those, 1133 subjects were randomized (567 to ponesimod 20 mg and 566 to teriflunomide 14 mg) across 162 sites in 28 countries, and 1131 subjects received at least one dose of study drug. The disposition of subjects is summarized in Table 1 and a summary of reasons (primary reason) for treatment discontinuation are shown in Table 2. Overall treatment and study discontinuation were balanced across both treatment arms. A total of 6.5% and 2.5% of the subjects discontinued due to AEs or tolerability related reasons in ponesimod 20 mg and teriflunomide 14 mg, respectively, while 1.9% and 4.3% discontinued due to efficacy related reasons. There were 2 deaths reported during the study—both on teriflunomide 14 mg.

### 1.1 Disposition and Treatment Discontinuation Information

TABLE 1
Disposition of subjects [Analysis Set - Subjects screened]

| | Ponesimod | | Teriflunomide | | | |
|---|---|---|---|---|---|---|
| | 20 mg | | 14 mg | | Total | |
| | N = 567 | | N = 566 | | N = 1133 | |
| | n (%) | | n (%) | | n (%) | |
| Subjects screened | | | | | 1468 | |
| Subjects re-screened | | | | | 110 | |
| Subjects randomized | 567 | (100) | 566 | (100) | 1133 | (100) |
| Subjects randomized after re-screening | 47 | (8.3) | 36 | (6.4) | 83 | (7.3) |
| Subjects treated | 565 | (99.6) | 566 | (100) | 1131 | (99.8) |
| Subjects completed treatment as per protocol | 471 | (83.1) | 473 | (83.6) | 944 | (83.3) |
| Subjects completed study as per protocol | 490 | (86.4) | 495 | (87.5) | 985 | (86.9) |
| Subjects completed treatment and | 465 | (82.0) | 465 | (82.2) | 930 | (82.1) |
| study per protocol | | | | | | |
| Subjects stayed in study beyond safety | 67 | (11.8) | 62 | (11.0) | 129 | (11.4) |
| follow-up (PTOP) | | | | | | |

Percentages based on subjects randomized Safety follow-up is up to EOT + 30 days.
PTOP = Post-treatment observation period.
Output: T_DS_02_SC, Produced by birdwi1 on 2019 Jul. 04T15:02 (CET), Data Extraction Date: 2019 Jun. 27, SDTM date: 2019 Jul. 03 Program: val_csr/program_output/T_DISP02.sas

TABLE 2
Reasons for premature treatment
discontinuation [Analysis Set - Safety Set]

| | Ponesimod | | Teriflunomide | | | |
|---|---|---|---|---|---|---|
| | 20 mg | | 14 mg | | Total | |
| | N = 565 | | N = 566 | | N = 1131 | |
| | n (%) | | n (%) | | n (%) | |
| Subjects who prematurely | 94 | (16.6) | 93 | (16.4) | 187 | (16.5) |
| discontinued study treatment | | | | | | |
| Reasons for premature | | | | | | |
| discontinuation of study treatment | | | | | | |
| Subject decision | 39 | (6.9) | 49 | (8.7) | 88 | (7.8) |
| Efficacy related | 7 | (1.2) | 14 | (2.5) | 21 | (1.9) |
| Tolerability related | 8 | (1.4) | 5 | (0.9) | 13 | (1.1) |
| Other | 19 | (3.4) | 26 | (4.6) | 45 | (4.0) |
| Not known | 5 | (0.9) | 4 | (0.7) | 9 | (0.8) |
| Physician decision | 40 | (7.1) | 23 | (4.1) | 63 | (5.6) |
| Adverse event | 29 | (5.1) | 9 | (1.6) | 38 | (3.4) |
| Lack of efficacy/treatment failure | 4 | (0.7) | 10 | (1.8) | 14 | (1.2) |
| Other | 7 | (1.2) | 4 | (0.7) | 11 | (1.0) |
| Pre-specified study treatment | 12 | (2.1) | 16 | (2.8) | 28 | (2.5) |
| discontinuation criteria | | | | | | |
| Lost to follow-up | 2 | (0.4) | 3 | (0.5) | 5 | (0.4) |
| Death | 0 | | 2 | (0.4) | 2 | (0.2) |
| Reason not provided | 1 | (0.2) | 0 | | 1 | (0.1) |

Output: T_DS_05_S, Produced by birdwi1 on 2019 Jul. 04T15:02 (CET), Data Extraction Date: 2019 Jun. 27, SDTM date: 2019 Jul. 03 Program: val_csr/program_output/DS05.sas

### 1.2 Demographic and Baseline Characteristics

Randomization was stratified by prior-DMT in the last two years prior to randomization (yes: 39.5%; no: 60.5%) and EDSS score at baseline (≤3.5: 83.3%; >3.5 16.7%). The mean age was 36.7 years and the majority of subjects (64.9%) were female. Most subjects were recruited in Europe with 50.6% from EU countries. Mean baseline EDSS score was 2.6, mean disease duration was 7.6 years and 97.4% were RRMS subjects. Mean pre-study 12-month relapse rate was 1.3, and 42.6% subjects had ≥1 Gd+ 1 lesions on brain MRI. The treatment arms were generally balanced in terms of demographics and baseline disease characteristics (Tables 3 and 4).

TABLE 3
Demographic characteristics [Analysis Set - Full Analysis Set]

| | Ponesimod | | Teriflunomide | | | |
|---|---|---|---|---|---|---|
| | 20 mg | | 14 mg | | Total | |
| | N = 567 | | N = 566 | | N = 1133 | |
| Sex [n (%)] | | | | | | |
| n | 567 | | 566 | | 1133 | |
| Male | 204 | (36.0) | 194 | (34.3) | 398 | (35.1) |
| Female | 363 | (64.0) | 372 | (65.7) | 735 | (64.9) |
| Age (years) | | | | | | |
| n | 567 | | 566 | | 1133 | |
| Mean | 36.7 | | 36.8 | | 36.7 | |
| SD | 8.74 | | 8.74 | | 8.74 | |
| Median | 36.0 | | 37.0 | | 37.0 | |
| Q1, Q3 | 30.0, | 44.0 | 30.0, | 44.0 | 30.0, | 44.0 |
| Min, Max | 18, | 55 | 18, | 55 | 18, | 55 |
| Race [n (%)] | | | | | | |
| n | 567 | | 566 | | 1133 | |
| White | 551 | (97.2) | 553 | (97.7) | 1104 | (97.4) |
| American Indian or | 0 | | 1 | (0.2) | 1 | (0.1) |
| Alaska Native | | | | | | |
| Black or African | 3 | (0.5) | 2 | (0.4) | 5 | (0.4) |
| American | | | | | | |
| Other | 5 | (0.9) | 2 | (0.4) | 7 | (0.6) |
| Not applicable | 8 | (1.4) | 8 | (1.4) | 16 | (1.4) |
| Geographical region/ | | | | | | |
| Country of enrolling | | | | | | |
| site [n (%)] | | | | | | |
| European Union | 289 | (51.0) | 284 | (50.2) | 573 | (50.6) |
| (EU) + UK | | | | | | |
| Europe Non-EU + | 233 | (41.1) | 239 | (42.2) | 472 | (41.7) |
| Russia | | | | | | |
| North America | 32 | (5.6) | 24 | (4.2) | 56 | (4.9) |
| Rest of World | 13 | (2.3) | 19 | (3.4) | 32 | (2.8) |

Output: T_DM_01_F (Modified from original), Produced by birdwi1 on 2019 Jul. 04T15:02 (CET), Data Extraction Date: 2019 Jun. 27, SDTM date: 2019 Jul. 03 Program: val_csr/program_output/DM01.sas

TABLE 4
Baseline disease characteristics [Analysis Set - Full Analysis Set]

| | Ponesimod | | Teriflunomide | | | |
|---|---|---|---|---|---|---|
| | 20 mg | | 14 mg | | Total | |
| | N = 567 | | N = 566 | | N = 1133 | |
| Baseline EDSS | | | | | | |
| N | 567 | | 566 | | 1133 | |
| Mean | 2.57 | | 2.56 | | 2.56 | |
| SD | 1.174 | | 1.229 | | 1.201 | |
| Median | 2.50 | | 2.50 | | 2.50 | |
| Q1, Q3 | 1.50, | 3.50 | 1.50, | 3.50 | 1.50, | 3.50 |
| Min, Max | 0.0, | 5.5 | 0.0, | 5.5 | 0.0, | 5.5 |
| Any DMT(a) received | | | | | | |
| within 2 years prior to | | | | | | |
| Randomization (eCRF) | | | | | | |
| [n (%)] | | | | | | |
| N | 567 | | 566 | | 1133 | |
| Yes | 213 | (37.6) | 211 | (37.3) | 424 | (37.4) |
| No | 354 | (62.4) | 355 | (62.7) | 709 | (62.6) |
| Time since first symptoms | | | | | | |
| (years) at randomization | | | | | | |
| N | 567 | | 566 | | 1133 | |
| Mean | 7.63 | | 7.65 | | 7.64 | |
| SD | 6.781 | | 6.782 | | 6.779 | |
| Median | 5.84 | | 5.70 | | 5.77 | |
| Q1, Q3 | 2.40, | 10.97 | 2.24, | 11.03 | 2.32, | 11.01 |
| Min, Max | 0.2, | 40.8 | 0.2, | 30.8 | 0.2, | 40.8 |
| Number of relapses in last | | | | | | |
| year prior to study entry | | | | | | |
| N | 567 | | 565 | | 1132 | |
| Mean | 1.2 | | 1.3 | | 1.3 | |
| SD | 0.61 | | 0.65 | | 0.63 | |
| Median | 1.0 | | 1.0 | | 1.0 | |
| Q1, Q3 | 1.0, | 1.0 | 1.0, | 2.0 | 1.0, | 1.0 |
| Min, Max | 0, | 4 | 0, | 5 | 0, | 5 |
| Multiple sclerosis subtype | | | | | | |
| [n (%)] | | | | | | |
| N | 567 | | 566 | | 1133 | |
| RRMS | 552 | (97.4) | 552 | (97.5) | 1104 | (97.4) |
| SPMS | 15 | (2.6) | 14 | (2.5) | 29 | (2.6) |
| Presence of Gd+ T1 lesions | | | | | | |
| at baseline (from central | | | | | | |
| reader) [n (%)] | | | | | | |
| N | 567 | | 564 | | 1131 | |
| Yes | 226 | (39.9) | 256 | (45.4) | 482 | (42.6) |
| No | 341 | (60.1) | 308 | (54.6) | 649 | (57.4) |
| Volume of T2 lesions at | | | | | | |
| baseline [mm3] (from | | | | | | |
| central reader) | | | | | | |
| N | 565 | | 563 | | 1128 | |
| Mean | 8301.4 | | 9489.2 | | 8894.3 | |
| SD | 10346.28 | | 11265.42 | | 10826.32 | |
| Median | 4841.3 | | 5651.0 | | 5171.7 | |
| Q1, Q3 | 1679.6, | 11004.4 | 2022.9, | 12978.7 | 1851.3, | 11754.1 |
| Min, Max | 0, | 86053 | 0, | 82776 | 0, | 86053 |
| Highly active disease | | | | | | |
| [n (%)] | | | | | | |
| N | 567 | | 566 | | 1133 | |
| Yes | 202 | (35.6) | 200 | (35.3) | 402 | (35.5) |
| No | 365 | (64.4) | 366 | (64.7) | 731 | (64.5) |

(a)DMT = MS disease-modifying treatment.
RRMS = Relapsing-remitting multiple sclerosis,
SPMS = Secondary progressive multiple sclerosis.
Output: T_SC_01_F (Modified from original), Produced by bridwi1 on 2019 Jul. 04T15:02 (CET), Data Extraction Date: 2019 Jun. 27, SDTM date: 2019 Jul. 03 Program: val_csr/program_output/SC01.sas

### 1.3 Extent of Exposure

The mean treatment exposure (irrespective of interruptions) was 96.7 weeks in the ponesimod 20 mg arm and 97.5 weeks in the teriflunomide 14 mg arm. The cumulative exposure to ponesimod 20 mg was 1045 subject-years and was 1057 subject-years for teriflunomide 14 mg arm.

TABLE 5
Study treatment exposure [Analysis Set - Safety Set]

| | Ponesimod | | Teriflunomide | |
|---|---|---|---|---|
| | 20 mg | | 14 mg | |
| | N = 565 | | N = 566 | |
| Treatment exposure, irrespective of interruptions (weeks) | | | | |
| N | 564 | | 566 | |
| Mean | 96.69 | | 97.45 | |
| SD | 29.018 | | 27.022 | |
| Median | 108.00 | | 108.00 | |
| Q1, Q3 | 107.29, | 108.71 | 107.29, | 108.57 |
| Min, Max | 0.3, | 111.3 | 0.1, | 113.0 |
| Treatment exposure, irrespective of interruptions | | | | |
| N | 564 | | 566 | |
| Cumulative exposure (years) | 1045.2 | | 1057.1 | |

Treatment exposure based on study drug log. Treatment duration only presented for subjects with available complete treatment end date. Interruptions derived based on study drug log and number of capsules taken. Output: T_EX_01_S(Modified from original), Produced by birdwil on 2019 Jul. 4T15:02 (CET), Data Extraction Date: 2019 Jun. 27, SDTM date: 2019 Jul. 3 Program: val_csr/program_output/EX01.sas

### 2. Safety

### 2.1 Summary of All Adverse Events

An overview of treatment emergent AEs (TEAEs) is presented in Table 6.

TABLE 6
Overview of treatment-emergent adverse
events (AE) [Analysis Set - Safety Set]

| | | |
|---|---|---|
| | Ponesimod | Teriflunomide |
| | 20 mg | 14 mg |
| | N = 565 | N = 566 |
| Characteristic | n (%) | n (%) |

| | | | | |
|---|---|---|---|---|
| Subject with at least one | | | | |
| AE | 502 | (88.8) | 499 | (88.2) |
| Severe AE | 39 | (6.9) | 26 | (4.6) |
| Drug-Related AE | 278 | (49.2) | 238 | (42.0) |
| AE leading to study drug | 49 | (8.7) | 34 | (6.0) |
| discontinuation | | | | |
| Serious AE | 49 | (8.7) | 46 | (8.1) |
| Fatal AE | 0 | | 2 | (0.4) |

OUTPUT: T_AE_01_S, Produced by JCD on 4 Jul. 2019 17:38 (CET), Data Extraction Date: 27 Jun. 2019, SDTM date: 3 Jul. 2019; Program: T_AE_01_S.sas

Overall, the proportion of subjects who experienced at least one TEAE was similar in both treatment arms (88.8% and 88.2% of subjects in the ponesimod 20 mg and the teriflunomide 14 mg arms, respectively).

The most common TEAEs in the ponesimod 20 mg arm were ALT increased (19.5%), nasopharyngitis (19.3%), headache (11.5%) and upper respiratory tract infection (10.6%). The most common TEAEs in the ponesimod 20 mg arm were ALT increased (19.5% vs 9.4% in the teriflunomide arm), nasopharyngitis (19.3% vs 16.8%), headache (11.5% vs 12.7%) and upper respiratory tract infections (10.6% vs 10.4%).

TEAEs leading to premature treatment discontinuation were reported in 8.7% of ponesimod 20 mg subjects compared to 6.0% of teriflunomide 14 mg subjects [see Table 7]. While the number of events was low, the difference in the type of AEs leading to treatment discontinuation was mainly driven by anticipated class effects on respiratory system and macular edema. No infections led to permanent study treatment discontinuation in the study.

TABLE 7
Treatment-emergent AEs leading to premature discontinuation
of study drug by SOC [Analysis Set - Safety Set]

| | Ponesimod | | Teriflunomide | |
|---|---|---|---|---|
| | 20 mg | | 14 mg | |
| | N = 565 | | N = 566 | |
| System Organ Class | n (%) | | n (%) | |
| Subjects with at least one AE | 49 | (8.7) | 34 | (6.0) |
| Investigations | 12 | (2.1) | 10 | (1.8) |
| Respiratory, thoracic | 7 | (1.2) | 0 | |
| and mediastinal disorders | | | | |
| Eye disorders | 5 | (0.9) | 0 | |
| Gastrointestinal disorders | 4 | (0.7) | 4 | (0.7) |
| Blood and lymphatic system disorders | 3 | (0.5) | 2 | (0.4) |
| General disorders and | 3 | (0.5) | 2 | (0.4) |
| administration site conditions | | | | |
| Hepatobiliary disorders | 3 | (0.5) | 2 | (0.4) |
| Pregnancy, puerperium and | 3 | (0.5) | 3 | (0.5) |
| perinatal conditions | | | | |
| Vascular disorders | 3 | (0.5) | 0 | |
| Nervous system disorders | 2 | (0.4) | 4 | (0.7) |
| Social circumstances | 2 | (0.4) | 1 | (0.2) |
| Cardiac disorders | 1 | (0.2) | 2 | (0.4) |
| Musculoskeletal and | 1 | (0.2) | 1 | (0.2) |
| connective tissue disorders | | | | |
| Neoplasms benign, malignant | 1 | (0.2) | 1 | (0.2) |
| and unspecified (incl | | | | |
| cysts and polyps) | | | | |
| Psychiatric disorders | 1 | (0.2) | 1 | (0.2) |
| Skin and subcutaneous tissue disorders | 1 | (0.2) | 2 | (0.4) |
| Reproductive system and | 0 | | 1 | (0.2) |
| breast disorders | | | | |
| Surgical and medical procedures | 0 | | 1 | (0.2) |

System Organ Classes are based on MedDRA version 21.0. SOCs are sorted by descending order of frequency in the ponesimod arm.
Modified from output T_AE_18_S, Produced by AGB on 4 Jul. 2019 17:38 (CET), Data Extraction Date: 27 Jun. 2019, SDTM date: 3 Jul. 2019, Program: T_AE_03_S_to_T_AE_23_1R.sas

There were two deaths reported in the study, one due to coronary artery insufficiency and one due to multiple sclerosis. Both deaths occurred in subjects receiving teriflunomide 14 mg.

The proportion of subjects who experienced at least one SAE was similar in both treatment arms (8.7% and 8.1% of subjects in the ponesimod 20 mg and the teriflunomide 14 mg arms, respectively).

An overview of AEs of special interest (AESIs) addressing anticipated risks of ponesimod is presented in Table 8. The most common AESIs were reported for category hepatobiliary disorders/liver enzyme abnormality (25.7% vs 14.5% in ponesimod 20 mg compared to teriflunomide 14 mg, respectively), followed by category hypertension (10.1% vs 9.0%), and pulmonary events (8.0% vs 2.7%).

TABLE 8
Treatment-emergent AESIs by category [Analysis Set - Safety Set]

| | Ponesimod | | Teriflunomide | |
|---|---|---|---|---|
| | 20 mg | | 14 mg | |
| | N = 565 | | N = 566 | |
| AESI Category | n (%) | | n (%) | |
| Hepatobiliary disorders/ | 145 | (25.7) | 82 | (14.5) |
| Liver enzyme abnormality | | | | |
| Hypertension | 57 | (10.1) | 51 | (9.0) |
| Pulmonary events | 45 | (8.0) | 15 | (2.7) |
| Effect on heart rate | 29 | (5.1) | 24 | (4.2) |
| and rhythm (including | | | | |
| hypotension) | | | | |
| Herpetic infection | 27 | (4.8) | 27 | (4.8) |
| Infection | 9 | (1.6) | 5 | (0.9) |
| Seizure | 8 | (1.4) | 1 | (0.2) |
| Macular edema | 6 | (1.1) | 1 | (0.2) |
| Skin malignancy | 5 | (0.9) | 1 | (0.2) |
| Non-skin malignancy | 1 | (0.2) | 1 | (0.2) |

Categories are sorted by descending order of frequency in the ponesimod 20 mg arm.
AESI—Adverse Event of Special Interest.
Infection AESI are identified by the AEs from the Infections and Infestations SOC, only if reported as serious or severe.
Modified from outputs T_AE_31_S, T_AE_38_S, T_AE_39_S, T_AE_41_S, T_AE_42_S, T_AE_43_S, T_AE_44_S, T_AE_45_S, T_AE_46_S, T_AE_48_S. All produced by JCD on 4 Jul. 2019 17:38.

The proportion of subjects who experienced ALT increase>3×ULN was higher in the ponesimod arm (17.3%) compared to teriflunomide (8.3%) whereas ALT increase>8×ULN was higher in the teriflunomide arm (2.1%) compared to ponesimod (0.7%). Based on the individual case review, most ALT/AST increases≥3×ULN occurred as a single transient asymptomatic episode, resolving with continued treatment or after protocol mandated treatment discontinuation. All but one case of bilirubin increase≥2×ULN occurred in subjects with pre-treatment bilirubin increases. One case of potential Hy's law occurred in a subject with pre-existing transaminase elevation (ALT>5×ULN), and the event fully resolved within 2 weeks after treatment discontinuation.

The incidence of treatment-emergent heart rate and rhythm (including hypotension) AESIs on Day 1 was higher in the ponesimod 20 mg arm (2.1%) than in the teriflunomide 14 mg arm (0.4%). See Table 8A. However, the overall incidence of first dose AESI on Day 1 was low (2.1%) in ponesimod. None of these events were serious nor led to permanent discontinuation of study treatment. Discharge criteria at 4 hours post-dose were met for ca. 99% of subjects. No 2nd or higher degree AV block was observed. ECG HR effect: nadir at 2 hours post-dose (siponimod—3-4 hours, fingolimod—around by 6 hours). Low incidence of low HR outliers (post-dose HR≤40 bpm), all 3 of them with a pre-treatment HR of <55 bpm, which is a known risk factor for post-dose bradycardia with S1P receptor modulators.

The mean heart rate reduction compared to pre-dose reached a maximum for ponesimod 20 mg at 2-hours post dose, −8.7 bpm compared to −1.7 bpm for teriflunomide 14 mg. There were 3 subjects with asymptomatic post-dose HR≤40 bpm in the ponesimod 20 mg arm (none on teriflunomide 14 mg); all of these subjects had a pre-treatment HR<55 bpm, which would require post-dose monitoring according to regulatory precedence of siponimod [Mayzent® USPI].

TABLE 8A
Treatment-emergent AESI by PT: Effect on heart rate and rhythm
(including hypotension) on Day 1 [Analysis Set - Safety Set]

| | Ponesimod | | Teriflunomide | |
|---|---|---|---|---|
| | 20 mg | | 14 mg | |
| | N = 565 | | N = 566 | |
| Preferred Term | n (%) | | n (%) | |
| Subjects with at least one AE | 12 | (2.1) | 2 | (0.4) |
| Bradycardia | 4 | (0.7) | 0 | |
| Atrioventricular block first degree | 3 | (0.5) | 0 | |
| Defect conduction intraventricular | 2 | (0.4) | 0 | |
| Bundle branch block left | 1 | (0.2) | 0 | |
| Bundle branch block right | 1 | (0.2) | 0 | |
| Sinus arrhythmia | 1 | (0.2) | 0 | |
| Sinus bradycardia | 1 | (0.2) | 0 | |
| Electrocardiogram QT prolonged | 0 | | 1 | (0.2) |
| Presyncope | 0 | | 1 | (0.2) |

Preferred Terms are based on MedDRA version 21.0.
Preferred terms are sorted by descending order of frequency in the ponesimod arm.
AESI—Adverse Event of Special Interest
OUTPUT: T_AE_32_S, Produced by JCD on 4 Jul. 2019 17:38 (CET), Data Extraction Date: 27 Jun. 2019, SDTM date: 3 Jul. 2019, Program: T_AE_32_S_and_T_AE_33_1R.sas

### Myelination Studies

### Introduction

The Phase III OPTIMUM study compared ponesimod (20 mg), a highly selective S1P1 receptor modulator, with teriflunomide (14 mg). Ponesimod treated MS patients showed a greater reduction in the annualized relapse rate, difference in Fatigue Symptoms and Impacts Questionnaire-Relapsing Multiple Sclerosis, reduction in active lesions/year, reduction in disability accumulation risk estimates and lower brain volume loss. The impact of treatment on myelination changes in normal appearing white matter (NAWM) which sustains damage early in the MS disease course was compared

### Objective

Compare ponesimod and teriflunomide treatment effects on changes in myelination.

### Methods

Myelination was estimated from T1 and T2-weighted magnetic resonance images collected at baseline, week 60 and week 108 in 427 patients treated with ponesimod and 428 with teriflunomide. Standardized T1/T2 was used to quantify myelination in non-lesional corpus callosum, cingulum, and remaining NAWM. Average myelination change (ΔM) from baseline at weeks 60 (w60) and 108 (w108) were compared across treatment arms using two-sided two sample t-tests, all p-values nominal.

### Results

With teriflunomide, myelination decreased significantly (p<0.05) across each of the 3 regions and at both visits. Conversely, in the ponesimod arm, only the corpus callosum showed a significant decrease at w108. At both visits, average myelination decreases were significantly larger for teriflunomide compared to ponesimod at all regions and both visits

For corpus callosum, the following was observed:

$${}_{w 60} Δ {M}^{pon} - {}_{w 60} Δ {M}^{ter} = 0.008 , p < 0.001 , {}_{w 108} Δ {M}^{pon} - {}_{w 108} Δ {M}^{ter} = 0.01 , p < 0.001 .$$

For cingulum, the following was observed:

$${}_{w 60} Δ {M}^{pon} - {}_{w 60} Δ {M}^{ter} = 0.0028 , p < 0.05 , {}_{w 108} Δ {M}^{pon} - {}_{w 108} Δ {M}^{ter} = 0.004 , p < 0.001 .$$

For remaining NAWM, the following was observed:

$${}_{w 60} Δ {M}^{pon} - {}_{w 60} Δ {M}^{ter} = 0.0025 , p < 0.05 , {}_{w 108} Δ {M}^{pon} - {}_{w 108} Δ {M}^{ter} = 0.004 , p < 0.001 .$$

### Conclusions

Ponesimod showed significantly less demyelination across different NAWM regions compared to Teriflunomide treated patients, indicating improved preservation of myelin and tissue microstructure.

1. A method of preserving myelination of axons in a human subject having a demyelinating disease, comprising administering to the subject in need thereof an effective amount of a monoselective S1P receptor modulator.

2. The method of claim 1, wherein the monoselective S1P receptor modulator is an S1P1 monoselective receptor modulator.

3. The method of claim 1, wherein the S1P1 monoselective receptor modulator is ponesimod.

4. The method of claim 1, wherein the demyelinating disease is multiple sclerosis, idiopathic inflammatory demyelinating disease, transverse myelitis, Devic's disease, progressive multifocal leukoencephalopathy, optic neuritis, leukodystrophy, Guillain-Barre syndrome, chronic inflammatory demyelinating polyneuropathy, autoimmune peripheral neuropathy, Charcot-Marie-Tooth disease, acute disseminated encephalomyelitis, adrenoleukodystrophy, adrenomyeloneuropathy, Leber's hereditary optic neuropathy, or HTLV-associated myelopathy.

5. The method of claim 4, wherein the demyelinating disease is multiple sclerosis.

6. The method of claim 5, wherein the multiple sclerosis is relapsing multiple sclerosis.

7. The method of claim 6, wherein the relapsing multiple sclerosis comprises relapsing-remitting disease, clinically isolated syndrome, or active secondary progressive disease.

8. The method of claim 7, wherein the relapsing multiple sclerosis comprises active secondary progressive disease.

9. The method of claim 1, wherein the human subject is treatment naïve.

10. The method of claim 1, wherein the human subject is at an early stage of disease progression and/or has experienced limited demyelination of axons.

11. The method of claim 1, wherein about 20 mg of ponesimod is administered orally once daily.

12. The method of claim 1, wherein the method comprises an up-titration step at initiation of the method or upon re-initiation of the method after a discontinuation, comprising administering orally once daily 2 mg of ponesimod on days 1 and 2; 3 mg of ponesimod on days 3 and 4; 4 mg of ponesimod on days 5 and 6; 5 mg of ponesimod on day 7; 6 mg of ponesimod on day 8; 7 mg of ponesimod on day 9; 8 mg of ponesimod on day 10; and 9 mg of ponesimod on day 11; 10 mg of ponesimod on days 12, 13, and 14, followed by the administering of the 20 mg of ponesimod once daily thereafter.

13. The method of claim 1, wherein the monoselective S1P receptor modulator is administered in the absence of a non-selective S1P receptor modulator or another monoselective S1P receptor modulator.

14. The method of claim 13, wherein the monoselective S1P receptor modulator is administered in the absence of a non-selective S1P receptor modulator.

15. The method of claim 13, wherein the monoselective S1P receptor modulator is administered in the absence of another monoselective S1P receptor modulator.

16. The method of claim 1, wherein the monoselective S1P receptor modulator is administered as a monotherapy.

17. A pharmaceutical product comprising ponesimod, wherein the pharmaceutical product is packaged and the package includes instructions for administering ponesimod to a human subject having a demyelinating disease in a regimen that is effective in preserving myelination of axons.

18-49. (canceled)


SEQUENCE LISTING (18 sequences)

SEQ ID NO: 1 - DNA, 23 bp, synthetic construct
  source 1..23: mol_type = other DNA
gcaacgatgtacgtctcttttgg

SEQ ID NO: 2 - DNA, 24 bp, synthetic construct
  source 1..24: mol_type = other DNA
gtacacaattcctttcttgtcatc

SEQ ID NO: 3 - DNA, 20 bp, synthetic construct
  source 1..20: mol_type = other DNA
ggctgtattcccctccatcg

SEQ ID NO: 4 - DNA, 21 bp, synthetic construct
  source 1..21: mol_type = other DNA
cagttggtaacaatgccatgt

SEQ ID NO: 5 - DNA, 22 bp, synthetic construct
  source 1..22: mol_type = other DNA
tcacagaagagaccctcacagc

SEQ ID NO: 6 - DNA, 20 bp, synthetic construct
  source 1..20: mol_type = other DNA
gagtcaaggatgcccgtgtc

SEQ ID NO: 7 - DNA, 22 bp, synthetic construct
  source 1..22: mol_type = other DNA
ttgtttgggaaaatggctagga

SEQ ID NO: 8 - DNA, 21 bp, synthetic construct
  source 1..21: mol_type = other DNA
gcagatggacagaaggttgga

SEQ ID NO: 9 - DNA, 19 bp, synthetic construct
  source 1..19: mol_type = other DNA
tccgtgcagaagtcgagaa

SEQ ID NO: 10 - DNA, 21 bp, synthetic construct
  source 1..21: mol_type = other DNA
ccatcactcaaaaggggtttc

SEQ ID NO: 11 - DNA, 20 bp, synthetic construct
  source 1..20: mol_type = other DNA
acacccccaacattgtggtt

SEQ ID NO: 12 - DNA, 20 bp, synthetic construct
  source 1..20: mol_type = other DNA
cgaactgcaaggtggtgttg

SEQ ID NO: 13 - DNA, 20 bp, synthetic construct
  source 1..20: mol_type = other DNA
tactcgcagggcaacaaaga

SEQ ID NO: 14 - DNA, 20 bp, synthetic construct
  source 1..20: mol_type = other DNA
cactgggttttcatccgtgc

SEQ ID NO: 15 - DNA, 21 bp, synthetic construct
  source 1..21: mol_type = other DNA
tccttctaccacctcagcgag

SEQ ID NO: 16 - DNA, 23 bp, synthetic construct
  source 1..23: mol_type = other DNA
ccggatggtcactctttaggaag

SEQ ID NO: 17 - DNA, 19 bp, synthetic construct
  source 1..19: mol_type = other DNA
ggatcgccagagccagcca

SEQ ID NO: 18 - DNA, 20 bp, synthetic construct
  source 1..20: mol_type = other DNA
gggcacaatagccccctcca